(19) United States
(12) Patent Application Publication
(10) Pub. No.: US 2007/0288012 A1
(43) Pub. Date: Dec. 13, 2007

(54) DYNAMIC MOTION SPINAL STABILIZATION SYSTEM AND DEVICE

(76) Inventors: Dennis Colleran, North Attleboro, MA (US); Arnold Oyola, Northborough, MA (US)

Correspondence Address: CARR LLP (IST), 670 FOUNDERS SQUARE, 900 JACKSON STREET, DALLAS, TX, 75202, US

(21) Appl. No.: 11/738,990

(22) Filed: Apr. 23, 2007

Related U.S. Application Data
(60) Provisional application No. 60/793,829, filed on Apr. 21, 2006.
(60) Provisional application No. 60/831,879, filed on Jul. 19, 2006.
(60) Provisional application No. 60/825,078, filed on Sep. 8, 2006.
(60) Provisional application No. 60/826,763, filed on Sep. 25, 2006.
(60) Provisional application No. 60/863,284, filed on Oct. 27, 2006.

(51) Int. Cl. A61B 17/58

(52) U.S. Cl. 606/61; 606/73

(57) ABSTRACT

Provided are a system and device for dynamically stabilizing a spine. In one example, the device includes one member having one end configured to rotatably couple to a bone anchor and another end having a curved channel. Another member of the device has one end configured to rotatably couple to another bone anchor and another end having a curved shaft positioned at least partially within the curved channel. A curvature of the curved channel and curved shaft restrains relative movement of the two members to a three dimensional curved surface.

### CLAIM OF PRIORITY

This application claims priority from U.S. Provisional Patent Application 60/793,829, entitled “Micro Motion Spherical Linkage Implant System,” filed on Apr. 21, 2006; U.S. Provisional Patent Application 60/831,879, entitled “Locking Assembly,” filed on Jul. 19, 2006; U.S. Provisional Patent Application 60/825,078, entitled “Offset Adjustable Dynamic Stabilization System,” filed on Sep. 8, 2006; U.S. Provisional Patent Application 60/826,763, entitled “Alignment Instrument for Dynamic Spinal Stabilization Systems,” filed on Sep. 25, 2006; U.S. Provisional Patent Application 60/863,284, entitled “Alignment Instrument for Dynamic Spinal Stabilization Systems,” filed on Oct. 27, 2006; U.S. patent application Ser. No. 10/914,751, entitled “System and Method for Dynamic Skeletal Stabilization,” filed on Aug. 9, 2004; U.S. patent application Ser. No. 11/303,138, entitled “Three Column Support Dynamic Stabilization System and Method,” filed on Dec. 16, 2005; U.S. patent application Ser. No. 11/467,798, entitled “Alignment Instrument for Dynamic Spinal Stabilization Systems,” filed on Aug. 28, 2006; and U.S. patent application Ser. No. 11/693,394, entitled “Dynamic Motion Spinal Stabilization System,” filed on Mar. 29, 2007. All of the above applications are incorporated by reference herein in their entirety.

### FIELD OF THE INVENTION

This disclosure relates to skeletal stabilization and, more particularly, to systems and method for stabilization of human spines and, even more particularly, to dynamic stabilization techniques.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (bending to either left or right side) and vertical (twisting of the shoulders relative to the pelvis).

In flexing about the horizontal axis into flexion (bending forward or anterior) and extension (bending backward or posterior), vertebrae of the spine must rotate about the horizontal axis to various degrees of rotation. The sum of all such movement about the horizontal axis of produces the overall flexion or extension of the spine. For example, the vertebrae that make up the lumbar region of the human spine move through roughly an arc of 15° relative to its adjacent or neighboring vertebrae. Vertebrae of other regions of the human spine (e.g., the thoracic and cervical regions) have different ranges of movement. Thus, if one were to view the posterior edge of a healthy vertebrae, one would observe that the edge moves through an arc of some degree (e.g., of about 15° in flexion and about 5° in extension if in the lumbar region) centered about a center of rotation. During such rotation, the anterior (front) edges of neighboring vertebrae move closer together, while the posterior edges move farther apart, compressing the anterior of the spine. Similarly, during extension, the posterior edges of neighboring vertebrae move closer together while the anterior edges move farther apart thereby compressing the posterior of the spine. During flexion and extension the vertebrae move in horizontal relationship to each other providing up to 2-3 mm of translation.

In a normal spine, the vertebrae also permit right and left lateral bending. Accordingly, right lateral bending indicates the ability of the spine to bend over to the right by compressing the right portions of the spine and reducing the spacing between the right edges of associated vertebrae. Similarly, left lateral bending indicates the ability of the spine to bend over to the left by compressing the left portions of the spine and reducing the spacing between the left edges of associated vertebrae. The side of the spine opposite that portion compressed is expanded, increasing the spacing between the edges of vertebrae comprising that portion of the spine. For example, the vertebrae that make up the lumbar region of the human spine rotate about an axis of roll, moving through an arc of around 10° relative to its neighbor vertebrae throughout right and left lateral bending.

Rotational movement about a vertical axis relative is also natural in the healthy spine. For example, rotational movement can be described as the clockwise or counter-clockwise twisting rotation of the vertebrae during a golf swing.

In a healthy spine the inter-vertebral spacing between neighboring vertebrae is maintained by a compressible and somewhat elastic disc. The disc serves to allow the spine to move about the various axes of rotation and through the various arcs and movements required for normal mobility. The elasticity of the disc maintains spacing between the vertebrae during flexion and lateral bending of the spine thereby allowing room or clearance for compression of neighboring vertebrae. In addition, the disc allows relative rotation about the vertical axis of neighboring vertebrae allowing twisting of the shoulders relative to the hips and pelvis. A healthy disc further maintains clearance between neighboring vertebrae thereby enabling nerves from the spinal chord to extend out of the spine between neighboring vertebrae without being squeezed or impinged by the vertebrae.

In situations where a disc is not functioning properly, the inter-vertebral disc tends to compress thereby reducing inter-vertebral spacing and exerting pressure on nerves extending from the spinal cord. Various other types of nerve problems may be experienced in the spine, such as exiting nerve root compression in the neural foramen, passing nerve root compression, and ennervated annulus (where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to each in order to maintain space for the nerves to exit without being impinged upon by movements of the spine.

In one such procedure, screws are embedded in adjacent vertebrae pedicles and rigid rods or plates are then secured between the screws. In such a situation, the pedicle screws press against the rigid spacer which serves to distract the degenerated disc space thereby maintaining adequate separation between the neighboring vertebrae to prevent the vertebrae from compressing the nerves. Although the foregoing procedure prevents nerve pressure due to extension of the spine, when the patient then tries to bend forward (putting the spine in flexion), the posterior portions of at least two vertebrae are effectively held together. Furthermore, the lateral bending or rotational movement between the affected vertebrae is significantly reduced, due to the rigid connection of the spacers. Overall movement of the spine is reduced as more vertebras are distracted by such rigid spacers. This type of spacer not only limits the patient's movements, but also places additional stress on other portions of the spine, such as adjacent vertebrae without spacers, often leading to further complications at a later date.

In other procedures, dynamic fixation devices are used. However, conventional dynamic fixation devices do not facilitate lateral bending and rotational movement with respect to the fixated discs. This can cause further pressure on the neighboring discs during these types of movements, which over time may cause additional problems in the neighboring discs.

Accordingly, dynamic systems which approximate and enable a fuller range of motion while providing stabilization of a spine are needed.

### SUMMARY

In one embodiment, a dynamic stabilization device comprises first and second members. The first member has a first end configured to rotatably couple to a first bone anchor and a second end having a curved channel. The second member has a third end configured to rotatably couple to a second bone anchor and a fourth end having a curved shaft slideably positioned at least partially within the curved channel. A curvature of the curved channel and curved shaft restrains movement of the first member relative to the second member to a three dimensional curved surface.

In still another embodiment, a method comprises identifying a center of rotation between first and second vertebrae. First and second alignment members are moveably coupled to first and second bone anchors, respectively. A first member of a dynamic stabilization device is coupled to the first alignment member and a second member of the dynamic stabilization device is coupled to the second alignment member. A longitudinal axis of the first alignment member is oriented with the center of rotation, and a longitudinal axis of the second alignment member is oriented with the center of rotation. The first and second alignment members are secured relative to the first and second bone anchors, respectively, to maintain the orientation of the first and second axes with the center of rotation.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is a side view of an embodiment of a dynamic stabilization system;

FIG. 2A is a perspective view of one embodiment of a dynamic stabilization device that may be used in the dynamic stabilization system of FIG. 1;

FIG. 2B is a side view of the dynamic stabilization device of FIG. 2A;

FIG. 3 is a perspective view of one embodiment of a member of the dynamic stabilization device of FIG. 2A;

FIG. 4 is a perspective view of one embodiment of a member of the dynamic stabilization device of FIG. 2A;

FIG. 5A is a side view of the dynamic stabilization system of FIG. 1 in a neutral position;

FIG. 5B is a side view of the dynamic stabilization system of FIG. 1 in an extension position;

FIG. 5C is a side view of the dynamic stabilization system of FIG. 1 in a flexion position

FIG. 6A is a posterior perspective view of the dynamic stabilization system of FIG. 1 in a neutral position;

FIG. 6B is a posterior perspective view of the dynamic stabilization system of FIG. 1 in an extension position;

FIG. 6C is a posterior perspective view of the dynamic stabilization system of FIG. 1 in a flexion position;

FIG. 6D is a posterior perspective view of the dynamic stabilization system of FIG. 1 in a lateral bending position;

FIG. 6E is a posterior perspective view of the dynamic stabilization system of FIG. 1 in a rotation extension position;

FIG. 6F is a posterior perspective view of the dynamic stabilization system of FIG. 1 in a rotation flexion position;

FIG. 7 is a posterior perspective view of an alternative embodiment of a dynamic stabilization system in a neutral position;

FIG. 8 is a posterior perspective view of another embodiment of a dynamic stabilization system in a neutral position;

FIG. 9 is a perspective view of one component that may be used with some embodiments of the dynamic stabilization system of FIG. 1; and

FIG. 10 is a flowchart of one embodiment of a method for using a dynamic stabilization system.

### DETAILED DESCRIPTION

It is to be understood that the following disclosure provides many different embodiments, or examples, for implementing different features of the disclosure. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Certain aspects of the present disclosure provide dynamic stabilization systems, dynamic stabilization devices, and/or methods for maintaining spacing between consecutive neighboring vertebrae and stabilizing a spine, while allowing movement of the vertebrae relative to each other in at least two and preferably three axes of rotation. The neighboring vertebrae may be immediately next to each other or spaced from each other by one or more intervening vertebrae.

It is sometimes difficult to match a dynamic stabilization system with a particular patient's anatomical structure while ensuring that a minimum range of motion is available for the dynamic implant due to factors such as the variability of pedicle to pedicle distance in the lumbar spine. In certain embodiments, it may be desirable to have a dynamic stabilization system implanted at a neutral position that allows for a minimum available range of motion, while having the system aligned with a center of rotation that is placed, for example, at the 60-70% A-P marker of a vertebral body.

For instance, if a sliding dynamic stabilization system has to be extended to reach amply spaced pedicles, the system may not have sufficient engagement left for flexion (i.e., the system may reach the end of the sliding motion before full flexion is achieved). In order to have a predictable and consistent range of motion, it may be desirable to have the relative starting engagement be the same (e.g., neutral). This may also be desirable to ensure that dampening forces are consistent at both extremes of relative motion.

Accordingly, the following disclosure describes dynamic stabilization systems, devices, and methods for dynamic stabilization which may provide for adjustable distraction of the inter-vertebral space while still allowing a patient a substantial range of motion in two and/or three dimensions. Such a dynamic stabilization system may allow the vertebrae to which it is attached to move through a natural arc that may resemble an imaginary three dimensional surface such as a sphere or an ellipsoid. Accordingly, such a system may aid in permitting a substantial range of motion in flexion, extension, rotation, anterior-posterior translation and/or other desired types of natural spinal motion.

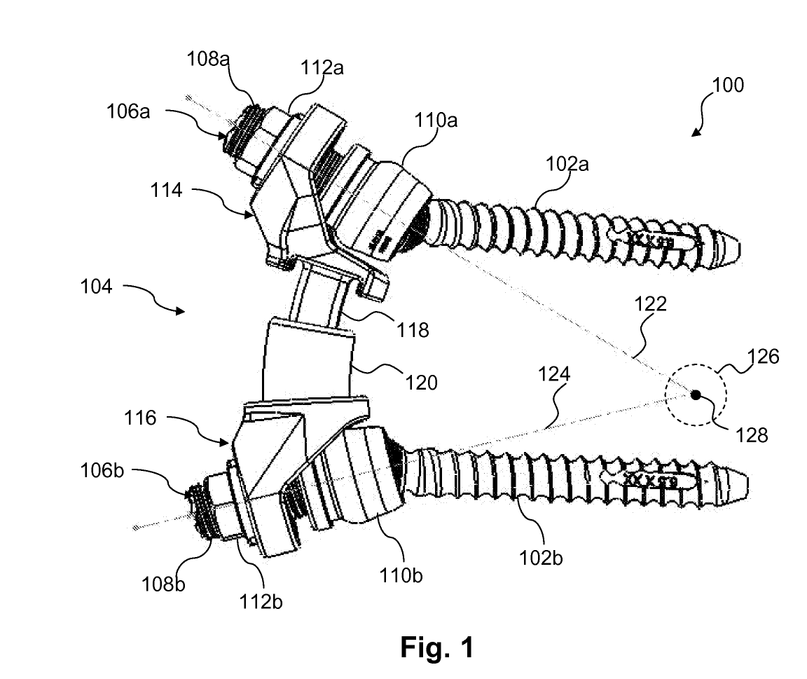

Referring to FIG. 1, there is illustrated one embodiment of a spine stabilization system 100. In the illustrated embodiment, the spine stabilization system 100 includes a plurality of bone anchors 102a and 102b which may be secured into a patient's vertebrae or other bone structures. The bone anchors 102a and 102b may be pedicle screws or other suitable bone anchoring devices known to those skilled in the art. A dynamic stabilization device 104 is coupled between the bone anchors 102a and 102b. The dynamic stabilization device 104 may be coupled to the bone anchors by threaded fastener systems 106a and 106b, which may enable adjustment of the dynamic stabilization device 104 relative to the bone anchors 102a and 102b. In certain embodiments, the dynamic stabilization device 104 may be adjusted so that relative movement between the exterior ends of the dynamic stabilization device follow the surface of a sphere or other three curved dimensional shape (e.g., an ellipsoid.

For example, portions of the threaded fastener systems 106a and 106b may be aligned with axes 122 and 124, respectively. The axes 122 and 124 may intersect an area 126 (e.g., an area of rotation). In some embodiments, the axes 122 and 124 may intersect at a point 128 (e.g., a center of rotation) within the area 126. The point 128 may be stationary or may move within the area 126 in conjunction with movement of the vertebrae (not shown) to which the spinal stabilization device 104 is coupled. It is understood that the area 126 and the point 128 are for purposes of illustration only and are not limited to the shapes or sizes shown. For example, while the area 126 is shown as a sphere, the area may be an ellipsoid or other shape. Furthermore, while the axes 122 and 124 are shown intersecting each other at the point 128, it is understood that they may not actually intersect one another, but may instead pass within a certain distance of each other. Furthermore, the point 128 need not be a stationary point, but may follow a path on or through the area 126. For example, the point 128 may move along a surface of the area 126 such that the area 126 provides a shell, and movement of the point 128 is constrained by the device 104 to an outer surface of the shell. For purposes of convenience, the term center of rotation may be used herein to refer to a specific point and/or a three dimensional surface.

The threaded fastener systems 106a and 106b may include alignment members or bearing posts (e.g., set screws) 108a and 108b received into polyaxial heads 110a and 110b that may be coupled to the proximal ends of the bone anchors 102a and 102b, respectively. As illustrated the bearing posts 108a and 108b may be independently adjusted with respect to the pedicle screws so that the longitudinal axis of the bearing posts may intersect with a center of rotation.

The fastener systems 106a and 106b may further include fasteners 112a and 112b for securing the dynamic stabilization device 104 to the bearing posts 108a and 108b. The fasteners 112a and 112b may be locking caps, nuts, or other similar threaded fasteners known to those skilled in the art. In some embodiments, the dynamic stabilization device 104 may rotate around one or both of the bearing posts 108a and 108b, while in other embodiments the dynamic stabilization device may be immovably fastened to the bearing posts.

The dynamic stabilization device 104 may include a male member 114 and a female member 116 each having an exterior and interior end. The male member 114 and female member 116 may be coupled together at their interior ends to allow for a sliding relative rotation about an axis of roll and a horizontal axis within a defined range of movement. The range of movement may be designed to permit a desired amount of lateral bending and twisting of upper and lower vertebrae relative to each other while maintaining a desired separation between the vertebrae. In certain embodiments, the male member 114 and female member 116 may be coupled by a curved shaft 118 of the male member 114 that is received into a channel of an extension 120 of the female member 116. In some embodiments, the curved shaft 118 may be sized to slideably move and/or rotate within the channel of the extension 120 about both a horizontal and vertical axis.

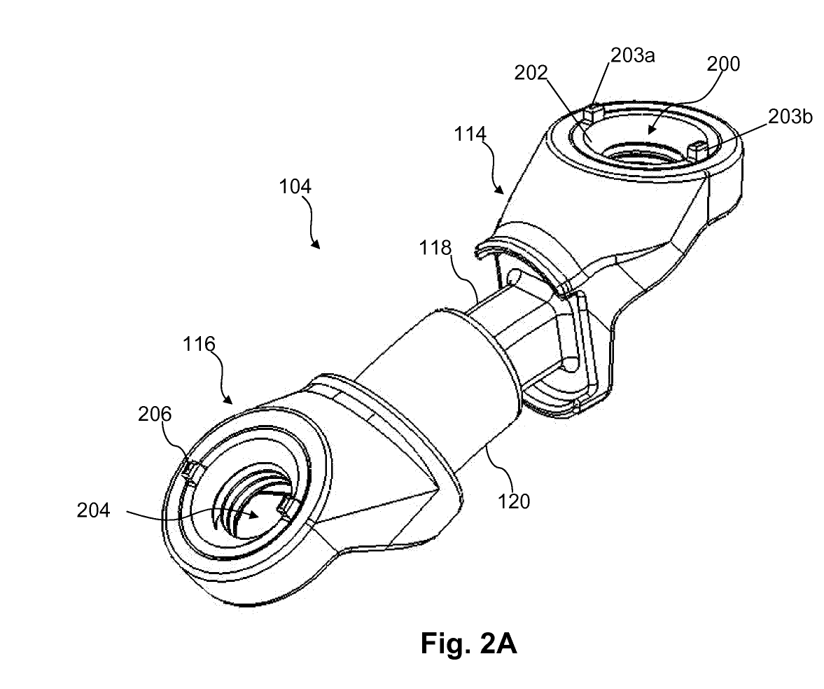

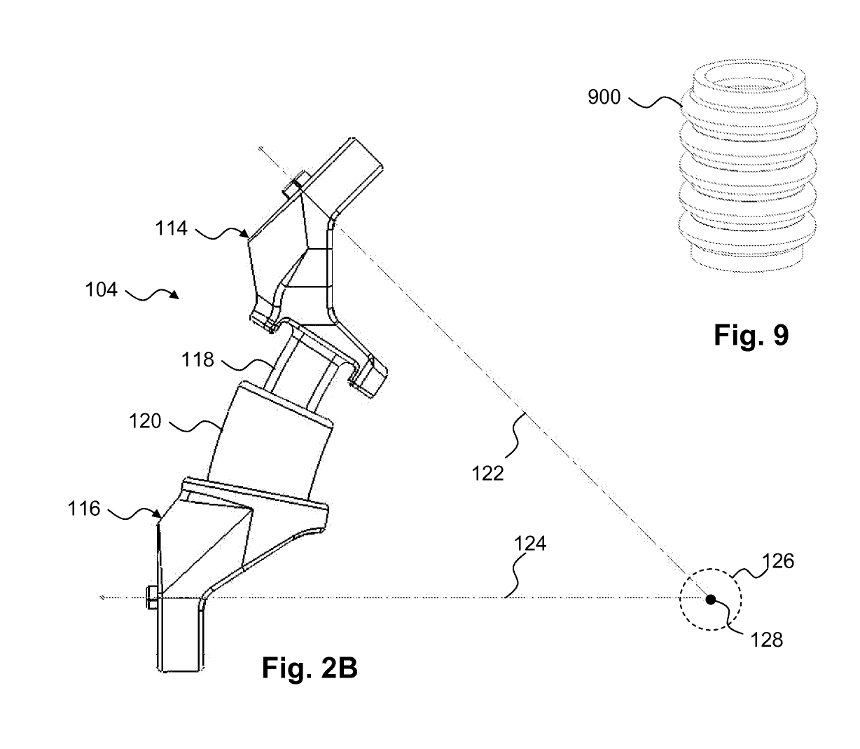

With additional reference to FIG. 2A, one embodiment of the dynamic stabilization device 104 is illustrated. In the present example, the male member 114 may include a threaded bearing or bushing 202 with an aperture 200 configured to receive the bearing post 108a of the threaded fastener system 106a (FIG. 1). The bushing 202 may have a plurality of gripping features 203a and 203b to hold and prevent the bushing from rotating while the bearing post 108a is inserted into the aperture 200. Alternatively, the bearing post 108a may be secured while the bushing 202 is rotated.

The bushing 202 may be inserted through the top of an opening located at one end of the male member 114. The bushing 202 may then be captured within the opening using a bushing cap (not shown) that is inserted from the bottom of the opening and secured (e.g., screw threads, press fit, welded) to the bushing 202. In some embodiments, an external surface of the bushing 202 or the bushing cap (not shown) may be relatively smooth or polished to facilitate rotation of the male member 114 around the bushing 202 when the system 106a is implanted. The bushing 202 or the bushing cap (not shown) may be manufactured from materials with good bearing properties such as cobalt chrome, stainless steel, titanium, UHMWPE, PEEK, carbon filled PEEK, or other biocompatible metals and polymers that are known in the art. The bearing post 108a may be secured to the bushing 202 by the fastener 112a.

The female member 116 may include an aperture 204 configured to receive the bearing post 108b of the threaded fastener system 106b (FIG. 1). A threaded bushing 206, which may be similar or identical to the threaded bushing discussed with respect to previous embodiments, may be positioned within the aperture 204. The bushing 206 may be secured in the aperture 204 using a bushing cap (not shown) that is secured (e.g., welded) to the bushing. In some embodiments, an external surface of the bushing 206 may be relatively smooth to facilitate rotation of the female member 116 around the bushing. The bearing post 108b may be secured to the bushing 206 by the fastener 112b.

Referring to FIG. 2B, a side view of the dynamic stabilization device 104 of FIG. 2A illustrates the male-female coupling relationship between the male member 114 and female member 116. As described previously, the extension 120 of the female member 116 may include a channel for receiving the curved shaft 118 of the male member 114 therein. For example, the curved shaft 118 may have a curved surface for slideably engaging one or more interior curved surfaces of the channel of the extension 120. This slideable engagement of the respective curved surfaces may allow the male member 114 and female member 116 to move relative to one another while maintaining their alignment with respect to the area of rotation 126 and/or center point 128. This may maintain the alignment of the dynamic stabilization device 104 with the spine's natural center of rotation, and may enable a more natural movement between the upper and lower vertebrae to occur while maintaining a degree of separation.

In certain embodiments, the curved shaft 118 and extension 120 may include horizontal curved surfaces that allow a slideable movement horizontally with respect to the center of rotation. If the radii of the vertical and horizontal curves of respective surfaces have a substantially similar or identical center or rotation, the male member 114 may move in a spherical manner with respect to the female member 116. In other words, the movement of the male member 114 and the female member 116 may follow a path that is constrained to a spherical surface (e.g., the area of rotation 126). It is understood that other curves may be used for the male member 114 and/or the female member 116 to create a non-spherical (e.g., ellipsoidal) path of movement.

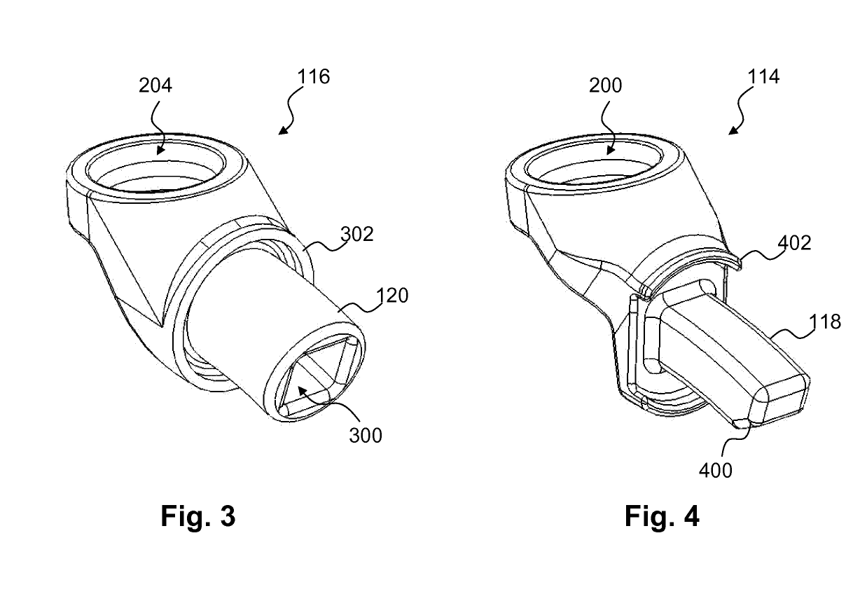

Referring to FIG. 3, a perspective view of one embodiment of the female member 116 of FIG. 1 is illustrated. In the present example, a channel 300 in the extension 120 is illustrated. As described previously, the channel 300 may be configured to receive the extension 118 of the male member 114. The channel 300 may be curved or straight, and may have any desired cross-sectional characteristics. For example, the illustrated channel 300 is substantially square in cross-section, but it is understood that the channel may have a cross-section that is circular, rectangular, or any other desired shape. A flange 302 may be formed around the extension 120 to engage or abut a complementary flange of the male member 114.

Referring to FIG. 4, a perspective view of one embodiment of the male member 114 of FIG. 1 is illustrated. As described previously, the curved shaft 118 may be configured to enter the channel 300 (FIG. 3) of the female member 116. While the shaft 118 is curved in the present example, it is understood that the shaft may be straight in some embodiments and may have any desired cross-sectional characteristics. For example, the illustrated curved shaft 118 is substantially square in cross-section, but it is understood that the shaft may have a cross-section that is circular, rectangular, or any other desired shape. In some embodiments, a distal portion of the curved shaft 118 may include a sloped surface 400. Such a surface 400 may, for example, aid movement of the curved shaft 118 within the channel 300. A flange 402 may be formed around the curved shaft 118 to engage or abut a complementary flange of the female member 116.

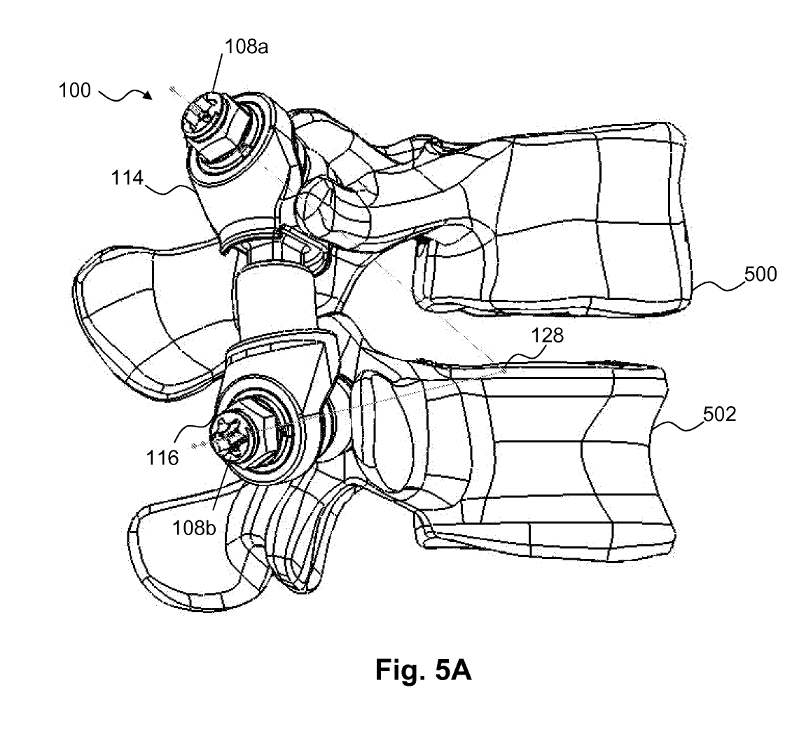

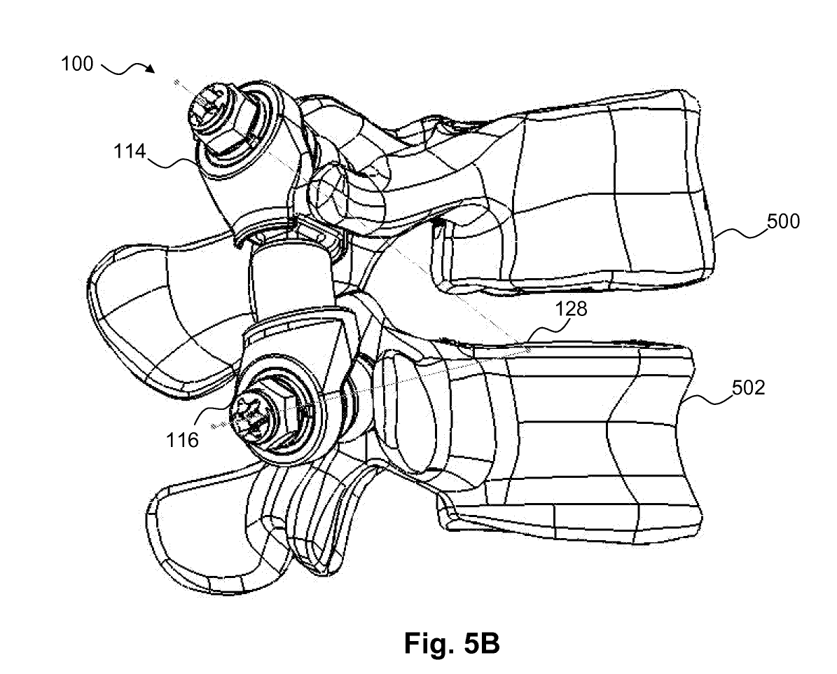

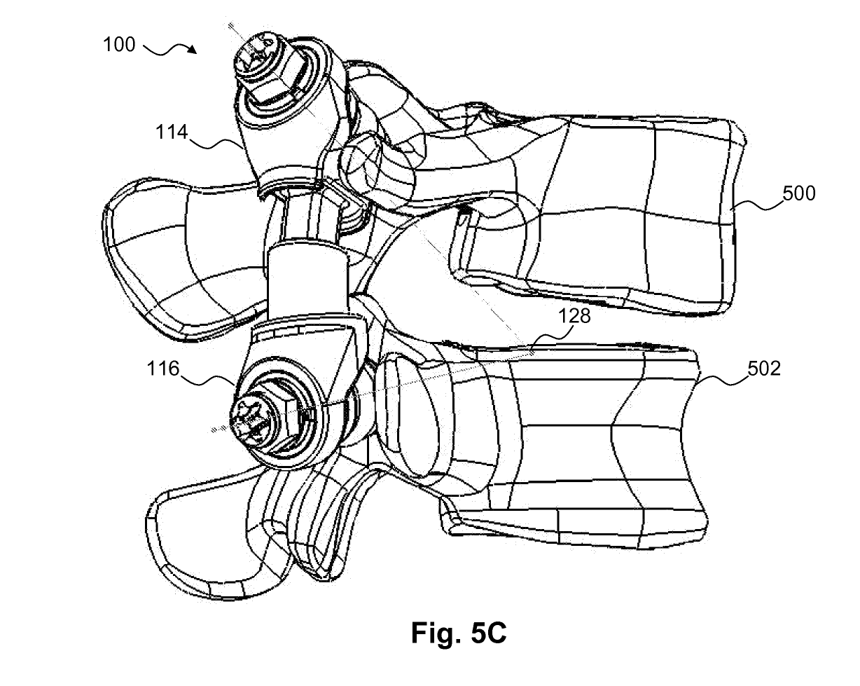

Referring to FIGS. 5A-5C, in one embodiment, side views illustrate the stabilization system 100 of FIG. 1 coupled to an upper vertebra 500 and a lower vertebra 502. As illustrated the bone anchors (not shown) are implanted into the respective vertebrae and the bearing posts 108a and 108b have been aligned such that their respective longitudinal axes point to a center of rotation 128. A similar alignment system (not shown) would also be implanted on the other side of the spine. The bearing posts of the other alignment system are also aligned so that their longitudinal axes point to the center of rotation 128. FIGS. 5A-5C also illustrate an exemplary range of motion and the center point 128 relative to the upper and lower vertebrae 500 and 502 around which the spine stabilization system 100 may rotate. FIG. 5A illustrates the spine stabilization system 100 when the two adjacent vertebrae 500 and 502 are in a neutral position. FIG. 5B illustrates the spine stabilization system 100 when the two adjacent vertebrae 500 and 502 are in a full extension position (e.g., when the patient is bending backward). FIG. 5C illustrates the spine stabilization system 100 when the two adjacent vertebrae 500 and 502 are in a flexion position (e.g., when the patient is bending forward).

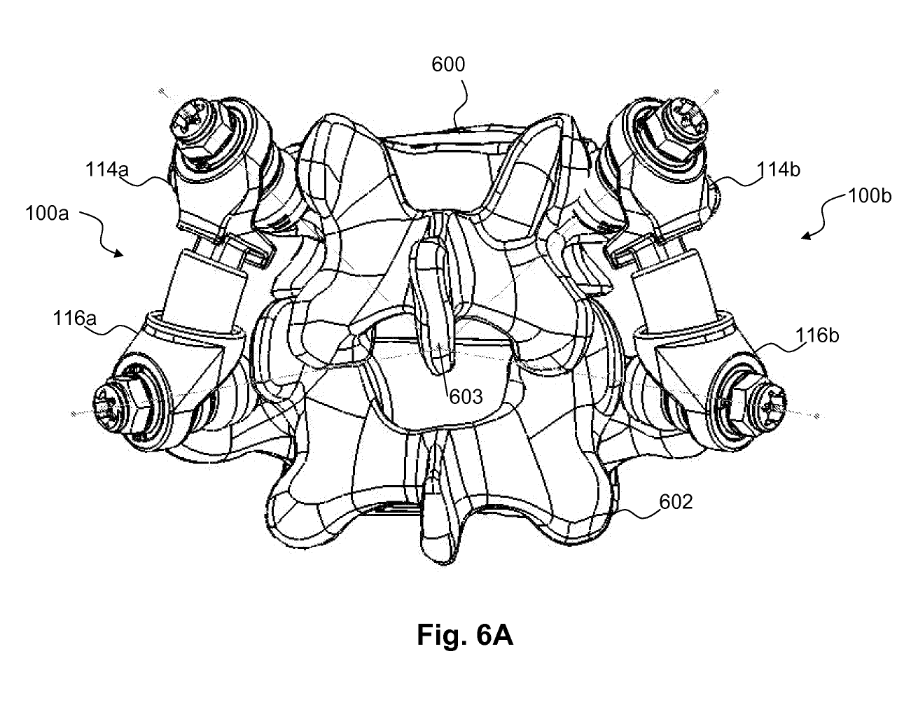

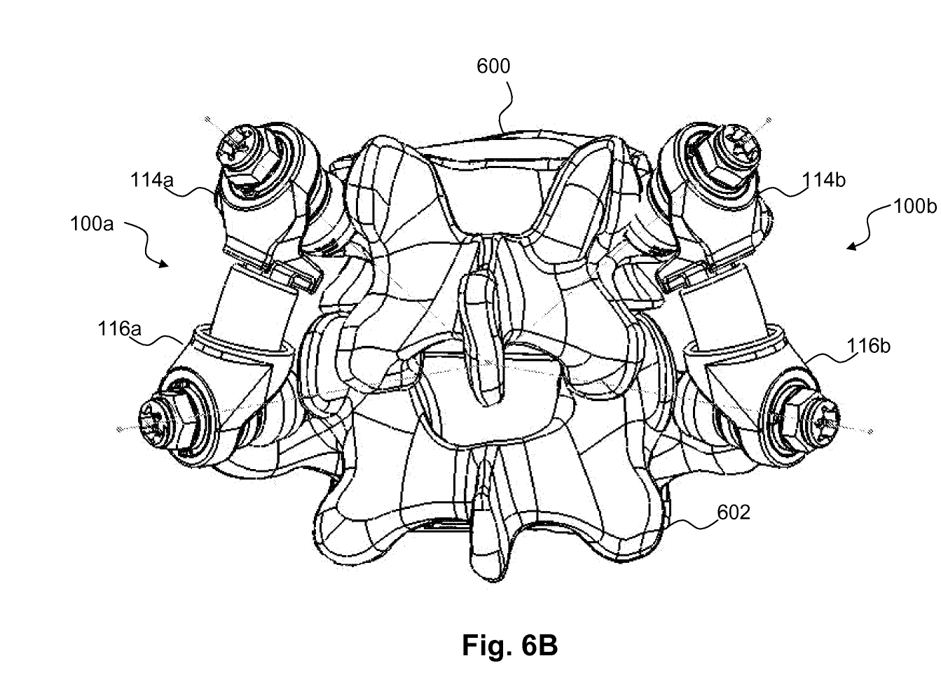

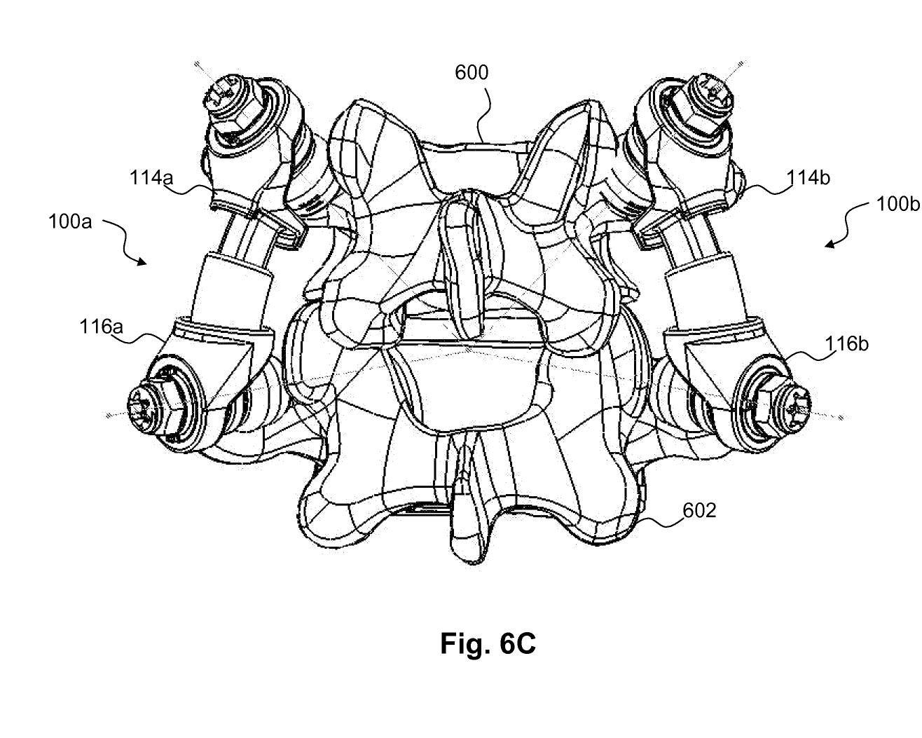

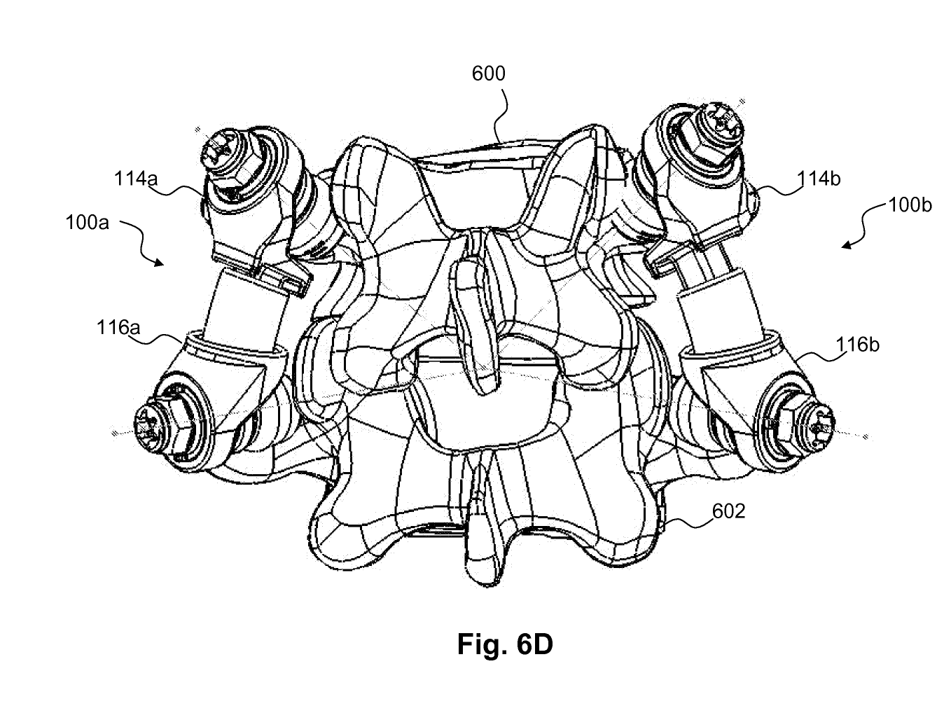

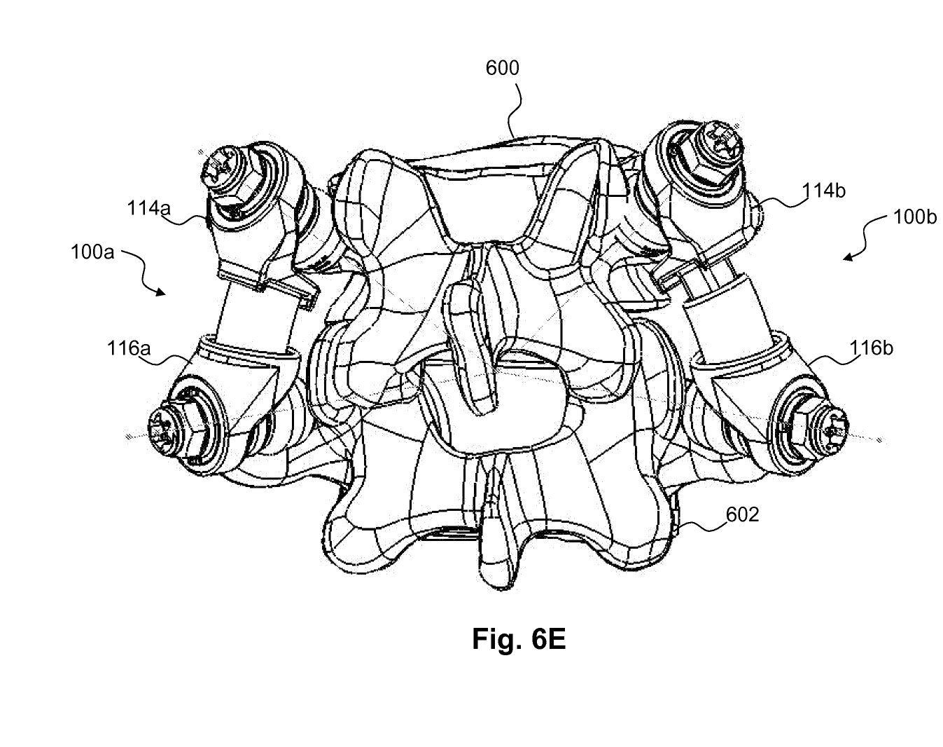

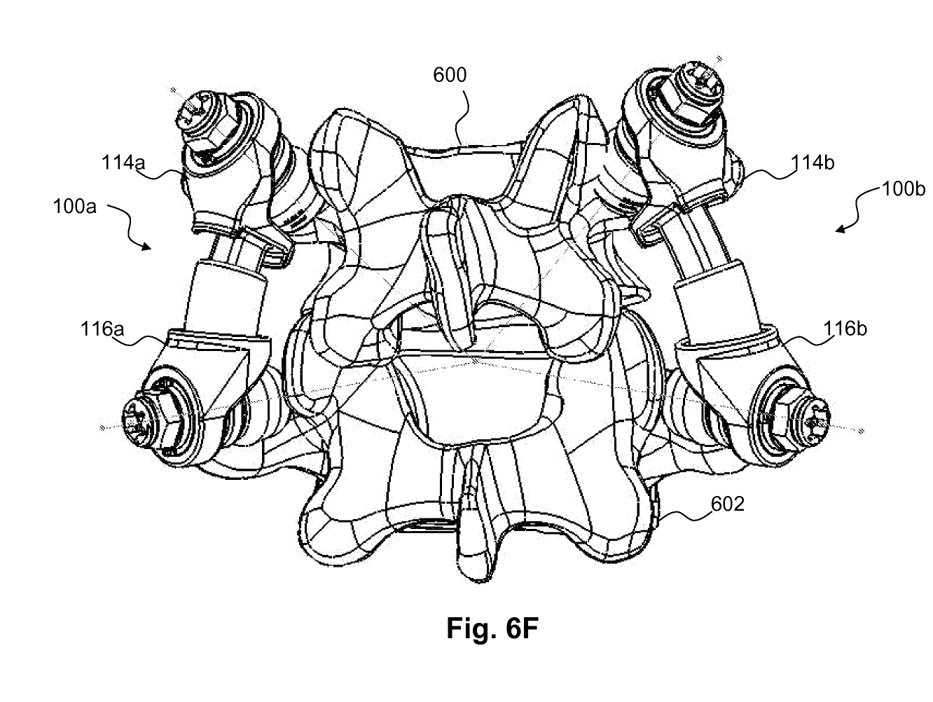

Referring to FIGS. 6A-6F, in one embodiment, posterior views illustrate two spine stabilization systems 100a and 100b coupled to an upper vertebra 600 and a lower vertebra 602. As illustrated, the bone anchors (not shown) of system 100a and 100b have been implanted into the respective vertebrae and each bearing posts of each system have been aligned such that their respective longitudinal axes point to a center of rotation 603. FIG. 6A illustrates the spine stabilization systems 100a and 100b when the two adjacent vertebrae 600 and 602 are in a neutral position. FIG. 6B illustrates the spine stabilization systems 100a and 100b when the two adjacent vertebrae 600 and 602 are in an extension position (e.g., when the patient is bending backward). FIG. 6C illustrates the spine stabilization systems 100a and 100b when the two adjacent vertebrae 600 and 602 are in a flexion position (e.g., when the patient is bending forward). FIG. 6D illustrates the spine stabilization systems 100a and 100b when the two adjacent vertebrae 600 and 602 are in a lateral bending position (e.g., when the patient is bending towards the right or left). FIG. 6E illustrates the spine stabilization systems 100a and 100b when the two adjacent vertebrae 600 and 602 are in a lateral rotational extension position (e.g., when the patient is turning and bending backward). FIG. 6F illustrates the spine stabilization systems 100a and 100b when the two adjacent vertebrae 600 and 602 are in a lateral rotational flexion position (e.g., when the patient is turning and bending forward).

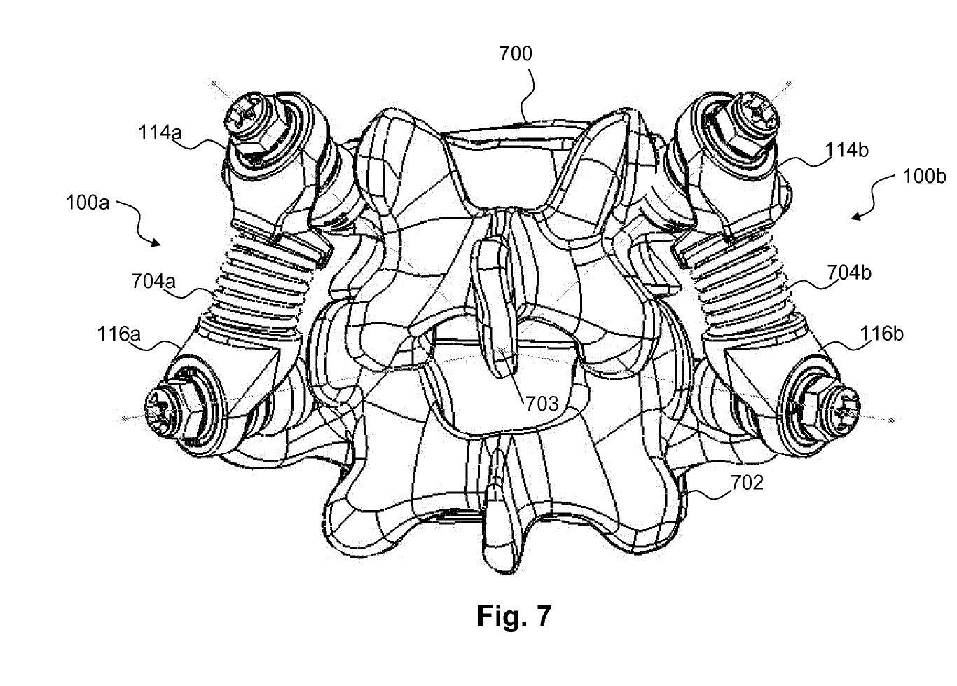

Referring to FIG. 7, in another embodiment, a posterior view is illustrated of the spine stabilization systems 100a and 100b when two adjacent vertebrae 700 and 702 are in a neutral position. As illustrated, the bone anchors (not shown) of system 100a and 100b have been implanted into the respective vertebrae and each bearing posts of each system have been aligned such that their respective longitudinal axes point to a center of rotation 703. In this example, the spine stabilization systems 100a and 100b incorporate control members 704a and 704b for controlling relative movement between the male members 114a and 114b and the respective female members 116a and 116b. In some embodiments, the control members 704a and 704b may be helical springs. The springs may provide an increasing resistance when the exterior ends of the male members 114a and 114b and the female members 116a and 116b slide closer together, such as in full extension. In some embodiments, the control members 704a and 704b may be coupled to both the male members 114a and 114b and the female members 116a and 116b. In such an embodiment, the control members 704a and 704b may also offer increasing resistance as the distance between the exterior ends of the male members 114a and 114b and the female members 116a and 116b increases, such as in full flexion.

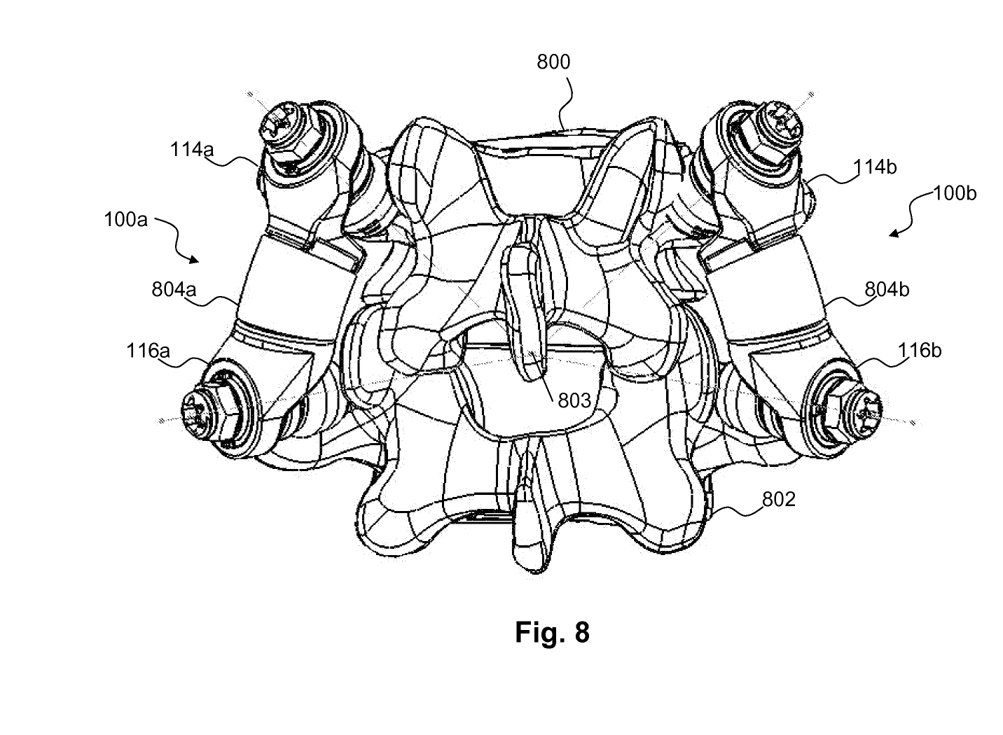

Referring to FIG. 8, in another embodiment, a posterior view illustrates two neighboring vertebrae 800 and 802 coupled to spine stabilization systems 100a and 100b. As illustrated, the bone anchors (not shown) of system 100a and 100b have been implanted into the respective vertebrae and each bearing posts of each system have been aligned such that their respective longitudinal axes point to a center of rotation 803. In this example, spine stabilization systems 100a and 100b incorporate control members 804a and 804b for controlling relative movement between the respective male members 114a and 114b and the female members 116a and 116b. In this embodiment, the control members 804a and 804b may be elastomeric sleeves. The control members 804a and 804b may provide an increasing resistance when the exterior ends of the male members 114a and 114b and the female members 116a and 116b slide closer together, such as in full extension. In some embodiments, the control members 804a and 804b may be coupled to both the male members 114a and 114b and the female members 116a and 116b. In such an embodiment, the control members 804a and 804b may also offer increasing resistance as the distance between the exterior ends of the male members 114a and 114b and the female members 116a and 116b increases, such as in full flexion. Furthermore, the sleeves may prevent surrounding flesh and tissue from intruding into the components of the respectively spine stabilization system.

Referring to FIG. 9, in yet another embodiment, a sleeve 900 is illustrated that may be used with embodiments of the spine stabilization systems discussed above. In this embodiment, the sleeve 900 may comprise a helical shape for use in conjunction with a spring member (not shown). In such embodiments, the spring may offer resistance or control the respective movement and the sleeve may prevent surrounding tissue from intruding into the spine stabilization system. In yet other embodiments, the sleeve may be made from a surgical mesh.

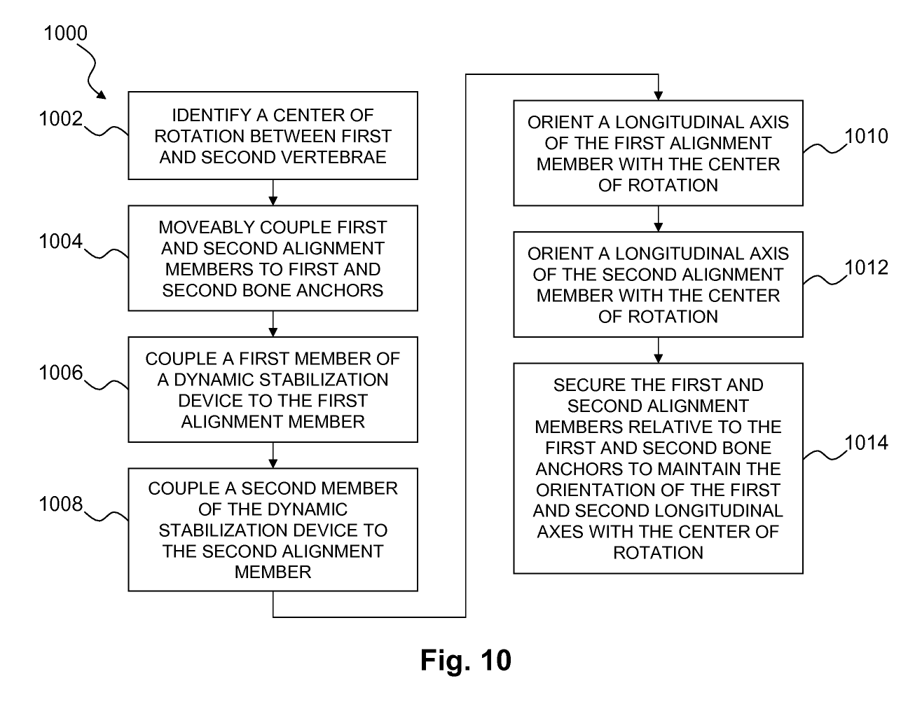

Referring to FIG. 10, in another embodiment, a method 1000 may be used to insert a dynamic stabilization system, such as the dynamic stabilization system 100 of FIG. 1. In step 1002, a center of rotation may be identified between first and second vertebrae. In step 1004, first and second alignment members (e.g., bearing posts) may be movably coupled to first and second bone anchors, respectively. For example, each alignment member may be screwed into a polyaxial head that is movably coupled to each bone anchor. In step 1006, a first member of a dynamic stabilization device may be coupled to the first alignment member and, in step 1008, a second member of the dynamic stabilization device may be coupled to the second alignment member. In steps 1010 and 1012, respectively, a longitudinal axis of each of the first and second alignment members may be oriented with the center of rotation. In step 1014, the first and second alignment members may be secured relative to the first and second bone anchors, respectively, to maintain the orientation of the first and second longitudinal axes with the center of rotation. For example, each alignment member may be tightened within its respective polyaxial head to abut the bone anchor and lock the polyaxial head's position relative to the bone anchor.

Although only a few exemplary embodiments of this disclosure have been described in details above, those skilled in the art will readily appreciate that many modifications are possible in the exemplary embodiments without materially departing from the novel teachings and advantages of this disclosure. Also, features illustrated and discussed above with respect to some embodiments can be combined with features illustrated and discussed above with respect to other embodiments. Accordingly, all such modifications are intended to be included within the scope of this disclosure.

What is claimed is:

1. A dynamic stabilization system comprising:
first and third alignment members coupled to first and third bone anchors, respectively, wherein each of the first and third bone anchors are affixed to a first vertebra;
second and fourth alignment members coupled to second and fourth bone anchors, respectively, wherein each of the second and fourth bone anchors are affixed to a second vertebra that is vertically spaced from the first vertebra;
a first dynamic stabilization device including:
a first member having a first end rotatably coupled to the first alignment member and a second end having a first curved channel; and
a second member having a third end rotatably coupled to the second alignment member and a fourth end having a first curved shaft positioned at least partially within the first curved channel, wherein a curvature of the first curved channel and first curved shaft restrains movement of the first member relative to the second member to a first three dimensional curved surface; and
a second dynamic stabilization device including:
a third member having a fifth end rotatably coupled to the third alignment member and a sixth end having a second curved channel; and
a fourth member having a seventh end rotatably coupled to the fourth alignment member and an eighth end having a second curved shaft positioned at least partially within the second curved channel, wherein a curvature of the second curved channel and the second curved shaft restrains movement of the third member relative to the fourth member to a second three dimensional curved surface.

2. The dynamic stabilization system of claim 1 wherein the first and the second three dimensional curved surfaces are the same three dimensional curved surface.

3. The dynamic stabilization system of claim 1 further comprising first, second, third, and fourth polyaxial heads coupled to the first, second, third, and fourth bone anchors, respectively.

4. The dynamic stabilization system of claim 3 wherein the first, second, third, and fourth alignment members include a bearing post threadably coupled to the first, second, third, and fourth polyaxial heads, respectively.

5. The dynamic stabilization system of claim 4 wherein first and second longitudinal axes of the first and second bearing posts, respectively, intersect a first center of rotation, and wherein third and fourth longitudinal axes of the third and fourth bearing posts, respectively, intersect a second center of rotation.

6. The dynamic stabilization system of claim 5 wherein the first and second centers of rotation are the same center of rotation.

7. The dynamic stabilization system of claim 5 wherein the curvature of the first curved channel and the first curved shaft maintains the intersection of the first and second longitudinal axes with the first center of rotation during movement of the first member relative to the second member.

8. A dynamic stabilization system comprising:
a first alignment member coupled to a first bone anchor, wherein the first alignment member includes a first longitudinal axis intersecting a center of rotation;
a second alignment member coupled to a second bone anchor, wherein the second alignment member includes a second longitudinal axis intersecting the center of rotation;
a first dynamic member having a first end configured to rotatably couple to the first alignment member and a second end having a curved channel; and
a second dynamic member having a third end configured to rotatably couple to the second alignment member and a fourth end having a curved shaft positioned at least partially within the curved channel, wherein a curvature of the curved channel and the curved shaft maintains an intersection of the first and second longitudinal axes with the center of rotation during movement of the first dynamic member relative to the second dynamic member.

9. The dynamic stabilization system of claim 8 further comprising first and second polyaxial heads coupled to the first and second bone anchors, respectively.

10. The dynamic stabilization system of claim 9 wherein the first and second alignment members are bearing posts threadably coupled to the first and second polyaxial heads.

11. A dynamic stabilization device comprising:
a first member having a first end configured to rotatably couple to a first bone anchor and a second end having a curved channel; and
a second member having a third end configured to rotatably couple to a second bone anchor and a fourth end having a curved shaft positioned at least partially within the curved channel, wherein a curvature of the curved channel and curved shaft restrains movement of the first member relative to the second member to a three dimensional curved surface.

12. The dynamic stabilization device of claim 11 wherein each of the first and third ends includes an aperture configured to receive a bearing post.

13. The dynamic stabilization device of claim 11 wherein the curved shaft and the curved channel are shaped to prevent the second member from rolling about a longitudinal axis of the curved channel.

14. The dynamic stabilization device of claim 13 wherein the curved channel and curved shaft are substantially rectangular in shape.

15. The dynamic stabilization device of claim 11 further comprising a control member positioned to exert force on the first and second members.

16. The dynamic stabilization device of claim 15 wherein the control member is an elastomeric sleeve coupled to the first and second members.

17. The dynamic stabilization system of claim 15 wherein the control member is a spring.

18. The dynamic member of claim 17 wherein the control member is coupled to the first and second members.

19. The dynamic stabilization device of claim 11 wherein first and second axes extending substantially perpendicularly through the first and third ends, respectively, intersect a center of rotation, and wherein the curvature of the curved channel and curved shaft maintains the intersection of the first and second axes with the center of rotation during movement of the first member relative to the second member.

20. A method comprising:
identifying a center of rotation between first and second vertebrae;
movably coupling first and second alignment members to first and second bone anchors, respectively;
coupling a first member of a dynamic stabilization device to the first alignment member;
coupling a second member of the dynamic stabilization device to the second alignment member;
orienting a longitudinal axis of the first alignment member with the center of rotation;
orienting a longitudinal axis of the second alignment member with the center of rotation; and
securing the first and second alignment members relative to the first and second bone anchors, respectively, to maintain the orientation of the first and second axes with the center of rotation.

21. The method of claim 20 wherein coupling the first member to the first alignment member includes locking a height of the first member relative to the first alignment member.

22. The method of claim 20 wherein movably coupling first and second alignment members to first and second bone anchors, respectively, includes threadably engaging first and second polyaxial heads, respectively, with the first and second alignment members.

23. The method of claim 22 wherein securing the first and second alignment members relative to the first and second bone anchors includes locking a position of the first and second polyaxial heads relative to the first and second bone anchors.
